(19) United States
(12) Patent Application Publication
(10) Pub. No.: US 2020/0230060 A1
(43) Pub. Date: Jul. 23, 2020

(54) SPRAY-DRIED WATER-SOLUBLE POWDER COMPOSITIONS AND PROCESSING METHODS THEREFOR

(71) Applicant: Bien Ventures Ltd., Calgary (CA)

(72) Inventors: Curtis Riley LEIFSO, Calgary (CA); Maria Bernice HAYAG, Calgary (CA); Andrew David WONG, Calgary (CA)

(21) Appl. No.: 16/376,733

(22) Filed: Apr. 5, 2019

(30) Foreign Application Priority Data
Jan. 21, 2019 (CA) 3030800

(51) Int. Cl. A61K 9/16; A61K 47/14; A61K 47/44; A23P 10/47; A23P 10/35

(52) U.S. Cl. CPC A61K 9/1623; A61K 9/1652; A61K 9/1682; A61K 47/14; A61K 47/44; A23V 2300/10; A23P 10/35; A23V 2002/00; A23V 2200/224; A23V 2300/26; A23P 10/47

(57) ABSTRACT

A composition for manufacturing a water-soluble powder via spray-drying. The composition has an emulsion and a core material dispersed therein. The emulsion has at least one carbohydrate membrane-forming material and a sugar alcohol mixed with a solvent. The core material at least has one or more active ingredients. After spray-drying, the water-soluble powder comprises the core material encapsulated by a membrane formed by the at least one carbohydrate membrane-forming material and the sugar alcohol.

### CROSS-REFERENCE TO RELATED APPLICATIONS

This application claims the benefit of Canadian Patent Application Serial No. 3,030,800, filed Jan. 21, 2019, the content of which is incorporated herein by reference in its entirety.

### FIELD OF THE DISCLOSURE

The present disclosure relates generally to spray-dried water-soluble powder compositions and processing methods therefor, and in particular to spray-dried water-soluble powder compositions with carbohydrates and a sugar alcohol as the membrane and corresponding processing methods therefor.

### BACKGROUND

Microencapsulation by spray-drying has been used for decades to encapsulate a wide range of active ingredients such as functional foods, essential oils, enzymes, lipids, flavors, pharmaceutical and cosmetic ingredients, and/or the like; see the academic paper entitled “Applications of Spray Drying in Microencapsulation of Food Ingredients: An Overview” by Gharsallaoui, A., Roudaut, G., Chambin, O., Voilley, A., and Saurel, R., published on Food Research International, 2007.

FIG. 1 shows a prior-art spray-drying microencapsulation process 10. After the process 10 starts (step 12), an emulsion is prepared (step 14). The emulsion generally comprises a mixture of a core comprising one or more active ingredients dispersed in a carrier oil, a wall material, and a solvent. At step 16, the prepared emulsion is then atomized via a pressurized nozzle into a plurality of droplets that are discharged into a high-temperature air stream to evaporate the solvent and obtain a spray-dried composition in the form of a powder.

FIG. 2A shows the structure of a droplet 40, in which the active ingredients 42 (which may be dispersed in a carrier oil) and solvent 44 are enclosed by the wall material 46. During the drying process, the wall material 46 forms a typically spherical, encapsulating membrane 48 (see FIG. 2B) enclosing the active ingredients 42 and some residual amount of unevaporated solvent 44.

The high-temperature air stream at the nozzle inlet substantially evaporates the solvent 44 in the droplets 40 and “dries” the droplets 40 to powder particles 40′ as shown in FIG. 2B. In an ideal case, the wall material 46 in a dried particle 40′ forms a matrix structure encapsulating the active ingredients 42 via a spherical encapsulating membrane 48. However, the prior-art particles 40′ often exhibit surface defects or surface anomalies that can adversely impact the oxidative stability thereof.

Referring back to FIG. 1, the process 10 ends (step 22) after the powder compositions 40′ are obtained. The resulting output from the drying process 10 produces water-soluble powders 40′ that may be rehydrated to yield the preserved core material including the active ingredients 42 for consumption.

In some prior-art embodiments, spray-dried powders 40′ obtained at step 16 may be further film-coated with a suitable material such as a surfactant for improving the solubility thereof (step 18). Moreover, it is common in prior art to add sweeteners and specifically sugar alcohols such as sorbitol to spray-dried powders 40′ as a post-processing bulking additive with the aim of improving flowability (step 20). The process 10 then ends (step 22).

The process 10 involves a careful evaluation of candidate materials to both augment the active ingredients 42 which are typically hydrophobic compounds, and the wall materials used to form the protective membrane 48 around the core 42 during the drying process.

Typically a polymer carbohydrate such as maltodextrins, gum acacia, or modified food starches may be used for forming the encapsulating membrane 48. Such polymer carbohydrates may also be used in combination to form the encapsulating membrane 48 in order to improve upon the generally poor interfacial properties of these materials when used stand-alone; see the academic paper entitled “Microencapsulation of Macadamia Oil by Spray Drying” by Laohasongkram, K., Mahamaktudsanee, T., and Chaiwanichsiri, S., and published on 11th International Conference on Engineering and Food, 2011.

Polymeric encapsulation of the core ingredients 42 offers many advantages including targeted delivery of Active Pharmaceutical Ingredients (APIs), water solubility, low moisture content, ease of handling and transport, protection of the core from oxidation and prolonged shelf life; see the academic paper entitled “Encapsulation Efficiency and Oxidative Stability of Flaxseed Oil Microencapsulated by Spray Drying Using Different Combinations of Wall Materials” by Carneiro, H., Tonon, R., Grosso, C., and Hubinger, M., and published on Journal of Food Engineering, 2012.

As described in the academic paper entitled “Recent Strategies in Spray Drying For the Enhanced Bioavailability of Poorly Water-Soluble Drugs” by Davis, M., and Walker, G., and published on Journal of Controlled Release, 2017, when the core material is an API, the emulsion preparation process (step 14) and drying parameters may be tailored to enhance the bioavailability of the core API either through targeted delivery whereby the membrane protects the core for a period of time as it passes through the gastrointestinal tract and/or by encapsulating micro or nano-sized emulsion droplets. Reducing the droplet size to nano-scale (typically on the order of 200 nanometers (nm)) has been shown to reduce API uptake time and to increase bioavailability as measured by blood plasma concentration; see the academic paper entitled “Influence of Droplet Size on Stability, in Vivo Digestion, and Oral Bioavailability of Vitamin E Emulsions” by Parthasarathi, S., Muthukumar, S., and Anandharamakrishnan, C., and published on Food and Function, 2016.

When a water-soluble powder is the desired end-product, solubility is a critical metric that the end user will benchmark the product against. Specifically, the rate at which the powders completely dissolve in water (or other medium), the thoroughness of dispersion and the amount of undesired residue and surface oil present after dissolution may be considered. Typical wall materials such as maltodextrin and gum acacia in combination may offer complete solubility when appropriate concentrations are used relative to the volume of the core material. However, the time required for complete dissolution is often longer than deemed acceptable by the user.

Solubility may be improved at the expense of added processing steps, additional ingredients or both. It is common to use fluid-bed technology to improve solubility by introducing the processing step 18 subsequent to spray-drying (step 16) for suspending the dried particles 40′ and applies a film-coating of a substance, often a surfactant such as lecithin, thereto. When the powder is dissolved, the film material alters the surface tension at the interface between the water and dried-particle membrane to facilitate rapid hydration of the particle and hence improve solubility. However, this process is both time and energy intensive while demanding specialized equipment.

An alternative processing method may be found in some commercial spray-dryers whereby fine particles with diameters below a given threshold are recirculated back into the inlet feed-stream thus increasing particle size through agglomeration. A larger particle size aids in submersing the particles on the water surface when the powder is dissolved which improves the solubility of the product. However, similar to the fluidizing bed, this process requires additional hardware and leads to relatively large mean particle-sizes which may not always be a desirable outcome.

To avoid the added time and cost associated with the above methods, it is desirable to improve solubility with an alternative emulsion formulation to be input into a conventional spray-dryer or a spray-dryer with minimal modification such as one which incorporates a multi-fluid nozzle. Several additive products such as Sodium Carboxymethyl Cellulose (CMC) and fumed silica (silicon dioxide) are known in prior art which may be mixed with the input emulsion or added into the drying-chamber inlet such that they are injected into the co-current feedstock/gas stream and bind to the dried particles.

While solubility may be improved with the addition of these additive substances, flowability is often the parameter most improved by such methods resulting in a product that still exhibits poor solubility. Moreover, these additional ingredients must be disclosed as part of the products' ingredients list and may not be desirable to consumers, particularly those conscientious about health and wellness or seeking naturally derived food ingredients.

Regardless of the method(s) chosen to improve solubility, other critical metrics of the dried powder should not be impaired to improve solubility. Such metrics include oxidative stability, particle size, morphology and flowability. Ideally, a desired solubility can be achieved with the introduction of low-cost ingredients into the emulsion feedstock that does not introduce ingredients deemed to be offensive to health-conscious consumers and does not require additional hardware or processing steps.

### SUMMARY

According to one aspect of this disclosure, there is provided a composition for manufacturing a water-soluble powder via spray-drying. The composition comprises an emulsion comprising at least one carbohydrate membrane-forming material and a sugar alcohol mixed with a solvent; and a core material dispersed in the emulsion, said core material at least comprising one or more active ingredients.

In some embodiments, the core material further comprises a carrier oil.

In some embodiments, the carrier oil is coconut oil.

In some embodiments, the carrier oil is medium-chain triglycerides (MCT) oil.

In some embodiments, the weight of the sugar alcohol is between about 20% to about 30% of the total weight of the at least one carbohydrate membrane-forming material, the sugar alcohol, and the core material.

In some embodiments, the sugar alcohol is xylitol, sorbitol, or a combination thereof.

In some embodiments, the at least one carbohydrate membrane-forming material comprises at least one of maltodextrin and modified starch.

According to one aspect of this disclosure, there is provided a composition for forming an emulsion to be mixed with one or more active ingredients for manufacturing a water-soluble powder via spray-drying. The composition comprises at least one carbohydrate membrane-forming material; and a sugar alcohol.

In some embodiments, the weight of the sugar alcohol is between about 33% to about 42% of the total weight of the at least one carbohydrate membrane-forming material.

In some embodiments, the sugar alcohol is xylitol, sorbitol, or a combination thereof.

In some embodiments, the at least one carbohydrate membrane-forming material comprises at least one of maltodextrin and modified starch.

According to one aspect of this disclosure, there is provided an emulsion for manufacturing a water-soluble powder via spray-drying. The emulsion comprises at least one carbohydrate membrane-forming material; a sugar alcohol; and a solvent.

In some embodiments, the emulsion further comprises a core material comprising at least one or more active ingredients.

In some embodiments, the core material further comprises a carrier oil.

In some embodiments, the carrier oil is coconut oil.

In some embodiments, the carrier oil is MCT oil.

In some embodiments, the weight of the sugar alcohol is between about 20% to about 30% of the total weight of the at least one carbohydrate membrane-forming material, the sugar alcohol, and the core material.

In some embodiments, the sugar alcohol is xylitol, sorbitol, or a combination thereof.

In some embodiments, the at least one carbohydrate membrane-forming material comprises at least one of maltodextrin and modified starch.

In some embodiments, the solvent is water.

According to one aspect of this disclosure, there is provided a water-soluble composition. The composition comprises a core material, said core material at least comprising one or more active ingredients; and a membrane encapsulating the core material, the membrane comprising at least one carbohydrate membrane-forming material and a sugar alcohol.

In some embodiments, the weight of the sugar alcohol is between about 33% to about 42% of the total weight of the at least one carbohydrate membrane-forming material.

In some embodiments, the sugar alcohol is xylitol, sorbitol, or a combination thereof.

In some embodiments, the at least one carbohydrate membrane-forming material comprises at least one of maltodextrin and modified starch.

According to one aspect of this disclosure, there is provided a method for manufacturing a water-soluble powder. The method comprises: preparing an aqueous emulsion using at least one carbohydrate membrane-forming material, a sugar alcohol, a core material, and a solvent, said core material at least comprising one or more active ingredients; homogenizing the aqueous emulsion; and spray-drying the emulsion for obtaining the water-soluble powder.

In some embodiments, the core material further comprises a carrier oil.

In some embodiments, the carrier oil is coconut oil.

In some embodiments, the carrier oil is MCT oil.

In some embodiments, said homogenizing the aqueous emulsion comprises homogenizing the aqueous emulsion using ultrasonic processing, a high-pressure homogenizer, or a high-shear mixer.

In some embodiments, the weight of the sugar alcohol is between about 20% to about 30% of the total weight of the at least one carbohydrate membrane-forming material, the sugar alcohol, and the core material.

In some embodiments, the sugar alcohol is xylitol, sorbitol, or a combination thereof.

In some embodiments, the at least one carbohydrate membrane-forming material comprises at least one of maltodextrin and modified starch.

In some embodiments, the solvent is water.

### BRIEF DESCRIPTION OF THE DRAWINGS

FIG. 1 is a flowchart showing a prior-art spray-drying microencapsulation process;

FIG. 2A is a schematic diagram showing the structure of a droplet of a prior-art emulsion having a prior-art polymer wall material;

FIG. 2B is a schematic diagram showing the structure of a particle “dried” from the droplet shown in FIG. 2A and through the prior-art spray-drying microencapsulation process shown in FIG. 1;

FIG. 3A is a schematic diagram showing the structure of a droplet of an emulsion having a core material, a membrane-forming material, a sugar alcohol, and a solvent, according to some embodiments of this disclosure;

FIG. 3B is a schematic diagram showing the structure of a particle “dried” from the droplet shown in FIG. 3A after the prior-art spray-drying microencapsulation process shown in FIG. 1;

FIG. 4 is a flowchart showing a spray-drying microencapsulation process, according to some embodiments of this disclosure;

FIGS. 5A and 5B are scanning electron microscope (SEM) images of particles shown in FIG. 3B obtained through the process shown in FIG. 4, showing the particle morphology thereof;

FIGS. 6A and 6B are SEM images of prior-art particles shown in FIG. 2B obtained through the prior-art process shown in FIG. 1, showing the particle morphology thereof and for comparison with that of the particles shown in FIGS. 5A and 5B;

FIG. 7A shows the oxidation induction time of the particles shown in FIGS. 5A and 5B obtained through an oxidation susceptibility test; and

FIG. 7B shows the oxidation induction time of the prior-art particles shown in FIGS. 6A and 6B obtained through an oxidation susceptibility test, for comparison with that shown in FIG. 7A.

### DETAILED DESCRIPTION

Embodiments disclosed herein relate to a composition and a processing method therefor. In particular, the composition is in the form of particles of naturally derived active ingredients microencapsulated in a soluble membrane with improved solubility, flowability, oxidative stability, and particle morphology. The processing method allows the manufacturing of the composition with a desired solubility using existing drying processes without alteration thereto or introducing additional equipment therefor. The processing method also has the advantage of preserving small dried-particle sizes (on the order of 2 micrometers (μm) in diameter) for drying systems that inherently output small particle-diameters. This can be desirable in specialized applications such as inhalable powders.

According to one aspect of this disclosure, an aqueous emulsion is first prepared. During a spray-drying process, the emulsion is atomized via a pressurized nozzle into a heated air stream having a plurality of droplets and is injected into the drying chamber of a spray dryer for evaporating the solvent and obtaining a spray-dried composition in the form of a powder.

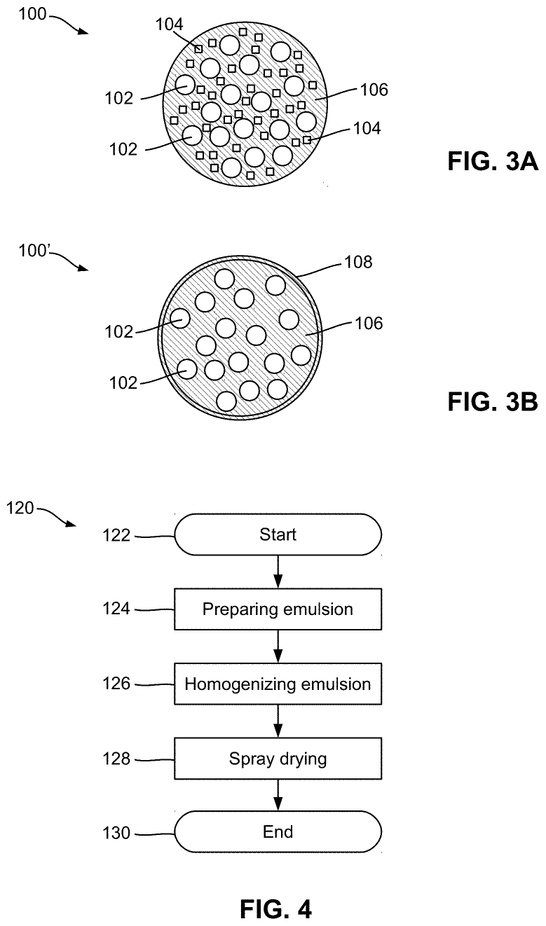

FIG. 3A shows the structure of a droplet 100 formed by the emulsion, according to some embodiments of this disclosure. As shown, the droplet 100 comprises a core material or composition 102, a solvent 104, and a membrane material or composition 106. The membrane composition 106 encloses the core composition 102 and the solvent 104, and forms and thickens a membrane during the spray-drying process (described in more detail later). Specifically, the membrane composition 106 forms a matrix structure engaging the core composition 102 and the solvent 104, and also forms a spherical encapsulating membrane 108 (see FIG. 3B) of increasing diameter during the spray-drying process enclosing the core composition 102 and the solvent 104.

FIG. 3B shows the structure of a particle 100′ dried from the droplet 100 after the solvent 104 therein is evaporated. Ideally the solvent 104 in the droplet 100 is completely evaporated during the drying process leaving only a small amount of residual moisture in the core encapsulated by the membrane 108.

In various embodiments, the core composition 102 comprises one or more active ingredients which may be any suitable compounds such as functional foods, essential oils, enzymes, lipids, flavors, pharmaceutical and cosmetic ingredients, and/or the like. For example, the active ingredients in some embodiments may be lipophilic compounds that are typically of a form including but not limited to powdered isolate, oils, concentrates, or other forms of extract. In some embodiments and depending on the types of the active ingredients, the core composition 102 may further comprise a carrier oil such as coconut oil or medium-chain triglycerides (MCT) oil (which is often derived from coconut oil) that may be used for dissolving lipophilic active-ingredients.

In various embodiments, the membrane composition 106 may comprise one or more membrane-forming materials and a sugar alcohol.

In some embodiments, the one or more membrane-forming materials may be one or more polymeric materials suitable for forming the particle wall or membrane 108 and/or for acting as emulsifying agent(s).

The one or more polymeric materials may be any carbohydrate materials suitable for forming the membrane 108. In some embodiments, the polymeric material may be maltodextrin, gum acacia, or modified starch. In some other embodiments, the polymeric material may be a combination of maltodextrin and modified starch.

In various embodiments, the sugar alcohol may be sorbitol, xylitol, and/or the like. In some embodiments, the sugar alcohol may be a combination of sorbitol, xylitol, and/or like. In some embodiments, the weight of the sugar alcohol may be between about 20% to about 30% of the total weight of the solids in the input emulsion (i.e., the total weight of the emulsion less that of the solvent therein) including the core composition 102 for a spray-drying process using a Buchi Mini Spray Dryer B-290 manufactured by BUCHI Labortechnik AG of Flawil, Switzerland, with a drying-inlet temperature of 150° C.

In some embodiments, the weight of the sugar alcohol may be between about 33% to about 42% of the total weight of the membrane composition 106 without the sugar alcohol. In some embodiments, the weight of the sugar alcohol may be between about 33% to about 42% of the total weight of the one or more membrane-forming materials. For example, in one embodiment where maltodextrin and modified starch are used as the membrane-forming materials, the weight of the sugar alcohol may be between about 33% to about 42% of the total weight of the maltodextrin and modified starch.

Those skilled in the art will understand that a suitable drying-inlet temperature is a function of the dryer size and construction, gas-flow rates, and emulsion feed rate. A same emulsion processed in different dryers may require different inlet temperatures for optimum product yields.

FIG. 4 shows a spray-drying microencapsulation process 120 according to some embodiments of this disclosure. The spray-dried powders obtained in accordance with the spray-drying microencapsulation process 120 facilitate nanoscale particle-sizes with desirable morphology aided by the high elasticity of the combination of the polymer membrane-forming materials and sugar alcohol. The spray-drying microencapsulation process 120 is particularly beneficial in pharmaceutical and nutraceutical preparations as it facilitates an enhancement of the bioavailability of the active ingredient and a faster uptake time of the obtained microencapsulated particles 100′ compared to those of the prior-art particles 40′.

After the spray-drying microencapsulation process 120 starts (step 122), an aqueous emulsion is prepared by mixing the above-described core composition 102, the membrane composition 106, and the solvent 104 (step 124).

At step 126, the prepared emulsion is homogenized for uniformly distributing the core composition 102 and the membrane composition 106 in the solvent 104 and for reducing the emulsion-droplet sizes such as reducing the mean droplet size in the emulsion to the order of 150 nm. Moreover, the homogenization of step 126 results in nano-sized active ingredients wherein the nano-sizing is preserved when the powder is rehydrated in an aqueous medium thereby improving the bioavailability of active ingredients that are for pharmaceutical application.

In one embodiment, the emulsion is homogenized using ultrasonic processing which applies an ultrasonic wave of suitable frequency and amplitude to the emulsion to impart cavitation for a suitable period of time. In another embodiment, the emulsion is homogenized using a high-pressure homogenizer. In yet another embodiment, the emulsion is homogenized using a high-shear mixer.

At step 128, the homogenized emulsion is atomized via a pressurized nozzle into a heated air stream into the drying chamber of a spray dryer for evaporating the solvent 104 and obtaining a spray-dried composition in the form of a powder. At this step, the drying-inlet temperature is controlled at a suitable temperature generally lower than the glass transition temperature of the composition materials in aggregate (including the core composition 102, the membrane-forming materials, and the sugar alcohol). As will be described in more detail later, the drying-inlet temperature and/or the concentration or weight percentage of the sugar alcohol in the emulsion may be controlled inversely for increased yields of the dried powders and/or for improved flowability and/or solubility thereof.

The process 120 ends (step 130) after the dried powders are obtained. The so-obtained dried powders have the advantageous properties of improved aqueous solubility, flowability and oxidative stability.

The above-described composition materials provide the microencapsulated particles 100′ with substantially greater water solubility compared to the microencapsulated particles 40′ manufactured using prior-art composition materials. In prior art, combinations of membrane-forming carbohydrates are used for forming the membrane encapsulating a lipophilic core that includes at least one active ingredient. While the resulting prior-art powder 40′ may exhibit complete water solubility if appropriate concentrations of constituent materials was used, the complete dissolution may take minutes which may be undesirably long when the powder is used in consumer beverage products.

In the embodiments disclosed herein, the solubility and flowability of the microencapsulated particles 100′ are increased via controlled introduction of a soluble low molecular-weight substance, which in some embodiments is the sugar alcohol, to reduce the molecular weight of the aggregate polymer matrix.

The molecular weight of a commonly used modified food starch is on the order of 9.6×10⁵ grams per mole (g/mol); see academic paper entitled “Characterizations of oil-in-water emulsion stabilized by different hydrophobic maize starches,” by Fan Ye, Ming Miao, Bo Jiang, Bruce R. Hamaker, Zhengyu Jin, and Tao Zhang, and published on Carbohydrate Polymers, Volume 166, Jun. 15, 2017, Pages 195-201. Maltodextrin with a high dextrose equivalence (DE=18) has a molecular weight on the order of 1017 g/mol, with a molecular weight varying inversely proportional to the dextrose equivalence; see academic paper entitled “Maltodextrin molecular weight distribution influence on the glass transition temperature and viscosity in aqueous solutions” by Avaltroni, F., Bouquerand, E., and Normand, V., and published on Carbohydrate Polymers, 2004.

Sugar alcohols such as sorbitol have a much lower molecular-weight than sugar at approximately 182 g/mol and exhibit excellent water solubility. In prior art, sugar alcohols are commonly used as bulking agents in a post-process in which the bulking agent is mixed with the dried powder and does not form an integral part of the membrane.

By forming an emulsion to be spray-dried that incorporates a suitable concentration of a sugar alcohol with respect to the membrane-forming materials and the core composition 102, the spray-dried particles 100′ obtained from the emulsion has a lower effective molecular-weight compared to the prior-art particles 40′.

Moreover, the effective molecular-weight of the spray-dried particles 100′ may be reasonably tailored in concert with the spray-drying conditions to alter the crystallinity of the dried matrix on account of the direct proportionate dependence of the glass transition temperature on the aggregate molecular weight.

In practice, the spray dryer's inlet temperature is set to not exceed the glass transition temperature of the composition materials in aggregate. Otherwise, when operated at an inlet temperature higher than the glass transition temperature, the powder output can be sticky and an unacceptably high amount of powder may adhere to the drying chamber surfaces thereby reducing the process yield.

In various embodiments, the range of input temperatures that will result in acceptable yields is very forgiving and allows a level of crystallinity as desired to increase or decrease solubility and flowability by varying the drying-inlet temperature, the sugar-alcohol content in the emulsion to be spray-dried, or both.

Specifically, flowability and/or solubility may be improved by either increasing the sugar-alcohol content for a given drying-inlet temperature, maintaining the sugar-alcohol content constant and increasing the drying-inlet temperature, or adjusting both the sugar-alcohol content and the drying-inlet temperature. While these adjustments may be limited by the requirements that the drying-inlet temperature shall not exceed the glass transition temperature and by the maximum sugar-alcohol concentration, the drying-inlet temperature and the sugar-alcohol concentration may still be adjusted within wide ranges for achieving an acceptable yield of microencapsulated particles 100′.

As described above, the weight of the sugar alcohol in some embodiments may be between about 20% to about 30% of the total weight of the solid content of the input emulsion (i.e., the total weight of the input emulsion less the total weight of the solvent in the emulsion).

For example, Table 1 below shows an exemplary formula for preparing the emulsion for spray-drying. As can be seen, the weight of the sugar alcohol is between about 20% to about 30% of the total weight of the carrier oil, active ingredient, membrane-forming materials (modified starch and the maltodextrin), and the sugar alcohol. The prepared emulsion is dried in a Buchi Mini Spray Dryer B-290 with a gas flow rate of 473 liters per hour (L/h), feed rate of 10 milliliters per minute (mL/min), a drying-inlet temperature of 150° C., and an outlet temperature of 69° C.

TABLE 1
Exemplary formula for forming the emulsion for spray-drying

| Input Material | Weight (g) |
|---|---|
| API + MCT Oil | 0.921 |
| Modified Food Starch | 3.85 |
| Maltodextrin | 7.736 |
| Sugar Alcohol | 3.1 to 4.7 |
| Solvent (Water) | 38.5 |

In various embodiments, improvement to both flowability and solubility may be achieved by using a range of combinations of a suitably low sugar alcohol weight-percentage with a suitably high drying-inlet temperature or by using a suitably high sugar alcohol weight-percentage with a suitably low drying-inlet temperature. On the other hand, if the sugar alcohol weight-percentage is too low and the drying-inlet temperature is too high, there would be insufficient sugar alcohol to impart the desired crystalline affect. If the sugar alcohol weight-percentage is too high and the drying-inlet temperature is too low, the resulting powder product would be too hygroscopic and exhibit an unacceptable sensitivity to moisture in open air as well as an unacceptably high moisture content post drying.

As sugar alcohols are miscible in water, the added sugar alcohol contributes to the quality of the formed membrane 108. In particular, the sugar alcohol is dissolved and re-dispersed to be integrated into the matrix where it serves to improve the viscoelastic properties of the membrane 108.

During the drying process (step 128 in FIG. 4), the membrane formation involves several stages of moisture removal from the core and thickening of the membrane. This process is facilitated by the elastic materials in the membrane-forming materials which facilitates moisture removal in tandem with improved particle morphology, resulting in uniform spherical particles 100′ free of surface defects that can otherwise adversely impact oxidative stability.

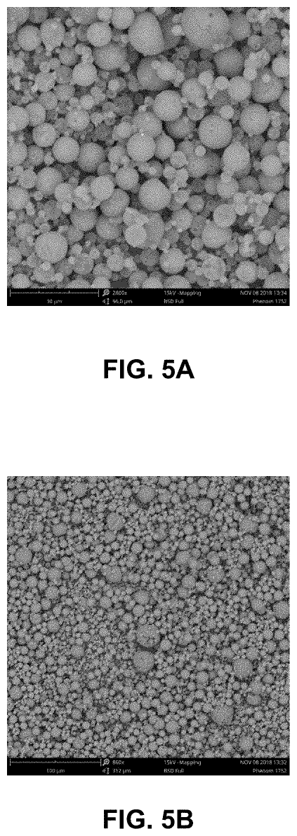

FIGS. 5A and 5B are scanning electron microscope (SEM) images of particles obtained from spray-drying the above-described emulsion having the membrane-forming material and the sugar alcohol (see FIG. 3A) through the spray-drying microencapsulation process 120 (see FIG. 4), showing the particle morphology thereof.

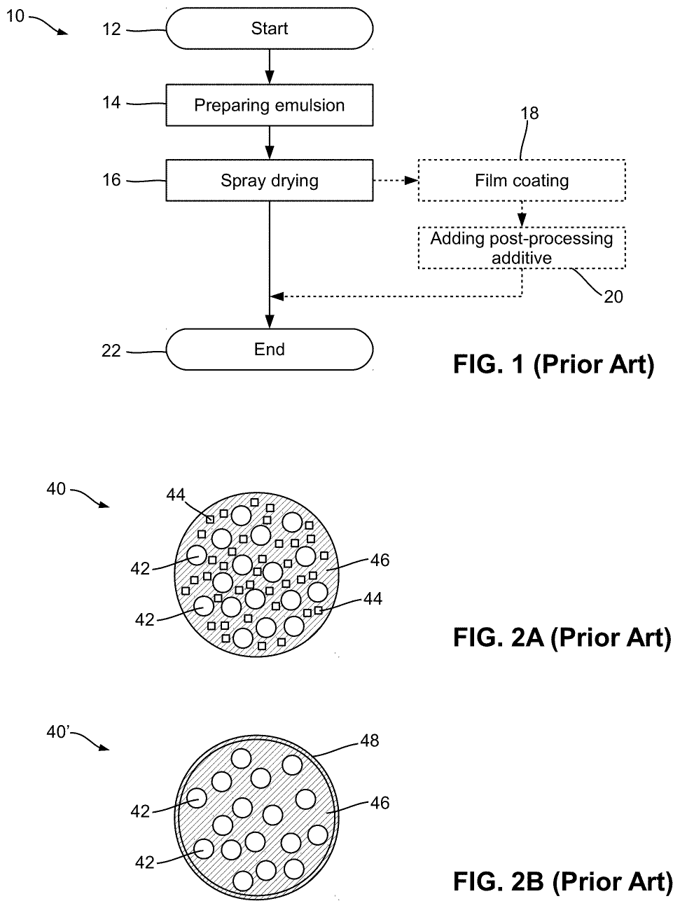

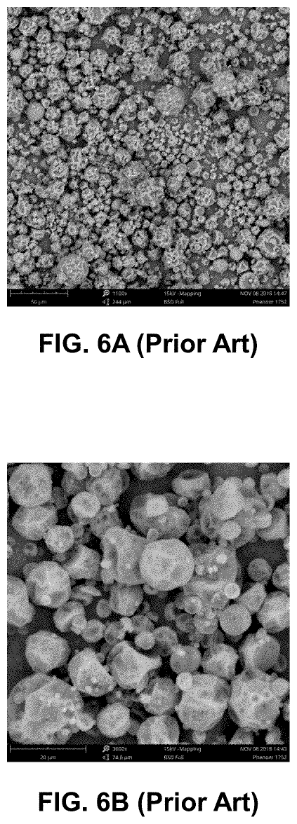

As a comparison, FIGS. 6A and 6B are SEM images of particles (denoted “prior-art particles” hereinafter) obtained from spray-drying a prior-art emulsion having no sugar alcohol (see FIG. 2A) through the prior-art spray-drying process 10 (see FIG. 1), showing the particle morphology thereof.

Compared to the prior-art particles shown in FIGS. 6A and 6B, the spherical particles shown in FIGS. 5A and 5B obtained from spray-drying the composition 100 through the spray-drying microencapsulation process 120 disclosed herein are free of defects or surface anomalies, thereby significantly avoiding the degraded oxidative stability that may be otherwise caused by such defects and/or surface anomalies.

An advantageous result of the improved morphology is the superior oxidative stability as the membrane is less susceptible to surface imperfections, blowholes and incomplete encapsulation that would otherwise undesirably allow oxidation of any exposed core. Oxidative stability tests described below illustrate this advantageous result.

Herein, accelerated ageing tests using the Rancimat method for characterizing the susceptibility to oxidation are conducted. The Rancimat method is described in the book entitled “Spray Drying Techniques for Food Ingredient Encapsulation” by Ishwarya, S., & Anandharamakrishnan, C., and published by John Wiley & Sons, Ltd. in 2015. By using the Rancimat method, a sample is placed in a reaction vessel at a constant temperature and is exposed to an airflow which causes oxidation to the sample (e.g., causing oxidation to the fatty acid thereof). The airflow transfers the volatile secondary oxidation products into a measuring vessel and are absorbed by a measuring solution such as distilled water. During the test, the electrical conductivity of the measuring solution is continuously recorded wherein increased conductivity indicates the appearance of the secondary oxidation products. The time to the detected appearance of the secondary oxidation products is then recorded as the induction time.

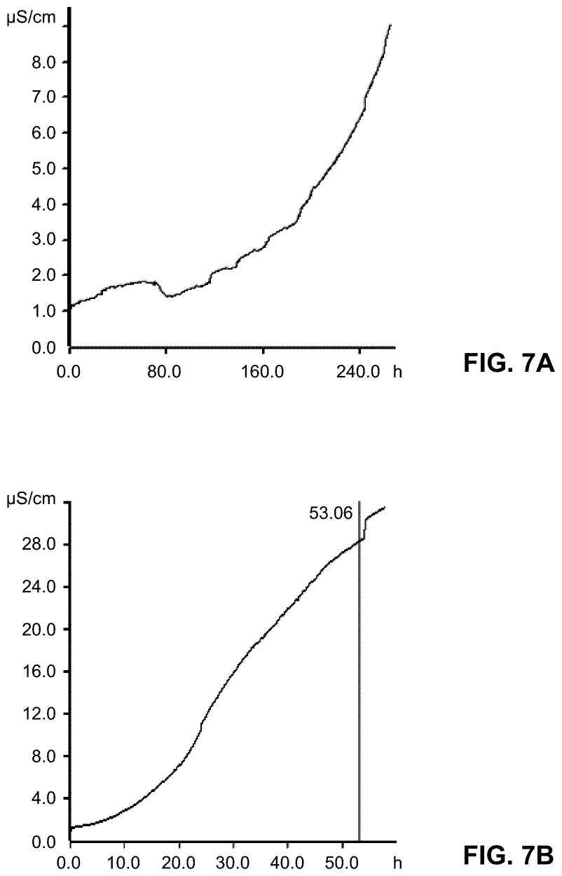

FIG. 7A shows the oxidation induction time of the particles shown in FIGS. 5A and 5B obtained through an accelerated ageing test using the Rancimat method. As a comparison, FIG. 7B shows the oxidation induction time of the prior-art particles shown in FIGS. 6A and 6B obtained through another accelerated ageing test using the Rancimat method.

As can be seen, with the addition of the sugar alcohol, the particles shown in FIGS. 5A and 5B exhibit no observed induction time (FIG. 7A) while the prior-art particles shown in FIGS. 6A and 6B (with no addition of sugar alcohol) exhibit a 53-hour induction time.

A further advantage of the composition and method disclosed herein is a significant improvement in powder flowability. Prior-art manufacturing employing various combinations of maltodextrin, gums, or starches usually forms a poorly flowing powder that complicates handling and packaging. On the other hand, in the composition and method disclosed herein, the addition of the sugar alcohol and tailoring of drying parameters facilitates an agglomeration of powder molecules that mitigates said sticking thereby improving handling and flowability.

Although embodiments have been described above with reference to the accompanying drawings, those of skill in the art will appreciate that variations and modifications may be made without departing from the scope thereof as defined by the appended claims.

What is claimed is:

1. A composition for manufacturing a water-soluble powder via spray-drying, the composition comprising:
an emulsion comprising at least one carbohydrate membrane-forming material and a sugar alcohol mixed with a solvent; and
a core material dispersed in the emulsion, said core material at least comprising one or more active ingredients.

2. The composition of claim 1, wherein said one or more active ingredients comprise at least one of functional foods, essential oils, enzymes, lipids, flavors, pharmaceutical and cosmetics.

3. The composition of claim 1, wherein the core material further comprises a carrier oil.

4. The composition of claim 3, wherein the carrier oil is coconut oil or medium-chain triglycerides (MCT) oil.

5. The composition of claim 1, wherein the weight of the sugar alcohol is between about 20% to about 30% of the total weight of the at least one carbohydrate membrane-forming material, the sugar alcohol, and the core material.

6. The composition of claim 1, wherein the sugar alcohol is xylitol, sorbitol, or a combination thereof.

7. The composition of claim 1, wherein the at least one carbohydrate membrane-forming material comprises at least one of maltodextrin and modified starch.

8. A water-soluble composition comprising:
a core material, said core material at least comprising one or more active ingredients; and
a membrane encapsulating the core material, the membrane comprising at least one carbohydrate membrane-forming material and a sugar alcohol.

9. The composition of claim 8, wherein said one or more active ingredients comprise at least one of functional foods, essential oils, enzymes, lipids, flavors, pharmaceutical and cosmetics.

10. The water-soluble composition of claim 8, wherein the weight of the sugar alcohol is between about 33% to about 42% of the total weight of the at least one carbohydrate membrane-forming material.

11. The water-soluble composition of claim 8, wherein the sugar alcohol is xylitol, sorbitol, or a combination thereof.

12. The water-soluble composition of claim 8, wherein the at least one carbohydrate membrane-forming material comprises at least one of maltodextrin and modified starch.

13. A method for manufacturing a water-soluble powder, the method comprising:
preparing an aqueous emulsion using at least one carbohydrate membrane-forming material, a sugar alcohol, a core material, and a solvent, said core material at least comprising one or more active ingredients;
homogenizing the aqueous emulsion; and
spray-drying the emulsion for obtaining the water-soluble powder.

14. The method of claim 13, wherein the core material further comprises a carrier oil.

15. The method of claim 14, wherein the carrier oil is coconut oil or medium-chain triglycerides (MCT) oil.

16. The method of claim 13, wherein said homogenizing the aqueous emulsion comprises:
homogenizing the aqueous emulsion using ultrasonic processing, a high-pressure homogenizer, or a high-shear mixer.

17. The method of claim 13, wherein the weight of the sugar alcohol is between about 20% to about 30% of the total weight of the at least one carbohydrate membrane-forming material, the sugar alcohol, and the core material.

18. The method of claim 13, wherein the sugar alcohol is xylitol, sorbitol, or a combination thereof.

19. The method of claim 13, wherein the at least one carbohydrate membrane-forming material comprises at least one of maltodextrin and modified starch.

20. The method of claim 13, wherein the solvent is water.
